(19) United States
(12) Patent Application Publication
(10) Pub. No.: US 2012/0097475 A1
(43) Pub. Date: Apr. 26, 2012

(54) HEARING AID TIP HAVING MULTIPLE SOUND PORTS

(76) Inventor: Daniel R. Schumaier, Elizabethton, TN (US)

(21) Appl. No.: 13/339,813

(22) Filed: Dec. 29, 2011

(51) Int. Cl. A61B 7/02

(52) U.S. Cl. 181/135

(57) ABSTRACT

A tip for use in a hearing assistance device has multiple sound ports and is dimensioned for placement in the ear canal of a user. The tip includes a channel portion for conducting acoustic energy which includes a proximal end, a distal end, and a central channel that extends between the proximal end and distal end. The tip further includes a tip portion having at least two sound ports in direct communication with the central channel of the channel portion to receive the acoustic energy therefrom. Each of the sound ports conducts acoustic energy from the central channel of the channel portion into the ear canal of the user.

### FIELD

The present invention relates generally to hearing aids. More particularly, the present invention relates to a tip having multiple sound ports for use with a hearing assistance device.

### BACKGROUND

There are many types of hearing assistance devices, also referred to herein as hearing aids, which vary in size, power, circuitry and features. One type of hearing aid is a behind-the-ear (BTE) hearing aid as shown in FIG. 1, which has a BTE housing 30 that contains a microphone, amplifier, and processing and control electronics. Thin-tube and receiver-in-the-canal (RIC) instruments are also a class of BTE hearing aids. Some other types of hearing aids are placed into the ear canal and completely or partially obstruct the ear canal. As shown in FIG. 1, a small tube 23, referred to as a “thin tube,” runs from the BTE housing 30 and into the ear canal of the user to deliver the amplified sound. A small sound emitting structure, referred to as a tip 2, is connected to the opposite end of the tube 23 and is placed into the user's ear canal. RIC models also use a BTE housing with a thin wire connected to the body of the BTE housing which runs to a receiver in the ear canal. RIC models also use tips attached to the receiver to transfer sound into the ear canal.

The tip 2 serves several purposes: (1) providing an acoustic seal to block unwanted noise interference, (2) directing sounds generated by the hearing aid device into the user's ear canal, (3) holding a thin tube or RIC in the ear canal, and (4) providing various levels of venting from open to closed. FIG. 9 shows a prior art tip 2 for use in a thin tube hearing aid, the features of which are well known in the art. In general, the tip 2 is a bud-like attachment for a hearing aid device, which is constructed to fit into the ear canal of a user. As shown in FIG. 9, a conventional tip 2 comprises a first end 4, which is spaced apart from a second end 6. A central channel 8 traverses the length of the tip 2, originating at the first end 4 and terminating in a sound port opening 10 at the second end 6. Conventional tips include only a single sound port opening 10.

In operation, the tip 2 is placed into the ear canal of the user such that the sound port opening 10 faces the user's eardrum. Acoustic energy (i.e., sound), originating from the hearing aid device, enters the tip 2 at the first end 4, travels along the central channel 8, and exits through the port 10 and into the ear canal of the user.

One recurring problem that plagues hearing aids, and thin tube and RIC hearing aids in particular, is that ear wax (i.e., cerumen) in the ear canal tends to build up and completely or partially block part of the sound port in the tip. This blockage may cause many problems while using the hearing aid apparatus, including impairing the sound quality or potentially making the hearing aid device unusable.

What is needed, therefore, is a hearing assistance device that mitigates the loss of sound output from the hearing aid due to sound port blockage in the tip.

### SUMMARY

The above and other needs are met by a tip for use in a hearing aid apparatus for imparting acoustic energy to a user for assistance in hearing. The tip is dimensioned for placement in an ear canal of the user. The tip includes a channel portion for conducting acoustic energy which includes a proximal end, a distal end, and a central channel that extends between the proximal end and distal end. The tip further includes a tip portion connected to the distal end of the channel portion that is dimensioned for placement in the ear canal of the user. The tip portion includes at least two sound ports in direct communication with the central channel of the channel portion to receive the acoustic energy therefrom. Each of the sound ports conducts acoustic energy from the central channel of the channel portion into the ear canal of the user.

In one preferred embodiment, the tip portion has a substantially hemispherical surface and includes a central sound port placed substantially along a longitudinal axis of the tip. A plurality of peripheral sound ports are disposed in the substantially hemispherical surface at a substantially equal radial spacing around the central sound port. The tip portion of this embodiment includes a plurality of peripheral sound port bores connecting the peripheral sound ports to the central channel of the channel portion at an angle with respect to a longitudinal axis of the tip. The tip also includes a central sound port bore connecting the central sound port to the central channel of the channel portion.

In another preferred embodiment, the tip portion has a substantially cylindrical surface and includes a central sound port placed substantially along a longitudinal axis of the tip. Four or more peripheral sound ports are disposed in the substantially cylindrical surface of the tip portion at a substantially equal radial spacing around the central sound port. The tip portion of this embodiment includes four peripheral sound port bores connecting the four peripheral sound ports to the central channel of the channel portion at an angle of about 90 degrees with respect to the longitudinal axis of the tip. The tip further includes a central sound port bore connecting the central sound port to the central channel of the channel portion.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 depicts a behind-the-ear hearing aid with various components positioned on and in the ear of a user;

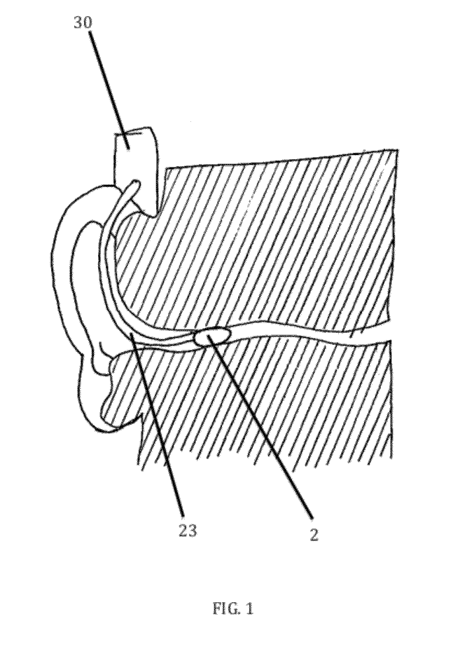

FIG. 2 is a perspective view of a first exemplary embodiment of a tip according to the present invention;

FIG. 3 is a front view of the tip shown in FIG. 2;

FIG. 4 is a sectional view of a first exemplary embodiment of the tip shown in FIG. 2;

FIG. 5 is a sectional view of a first exemplary embodiment of a tip, a connector member, and a thin tube according to the present invention;

FIGS. 6A and 6B are perspective views of a second exemplary embodiment of a tip according to the present invention;

FIG. 7 is a side view of a third exemplary embodiment of a tip according to the present invention;

FIGS. 8A, 8B and 8C depict a fourth exemplary embodiment of a tip; and

FIG. 9 depicts a prior art tip for a hearing aid apparatus.

### DETAILED DESCRIPTION

Various terms used herein are intended to have particular meanings Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

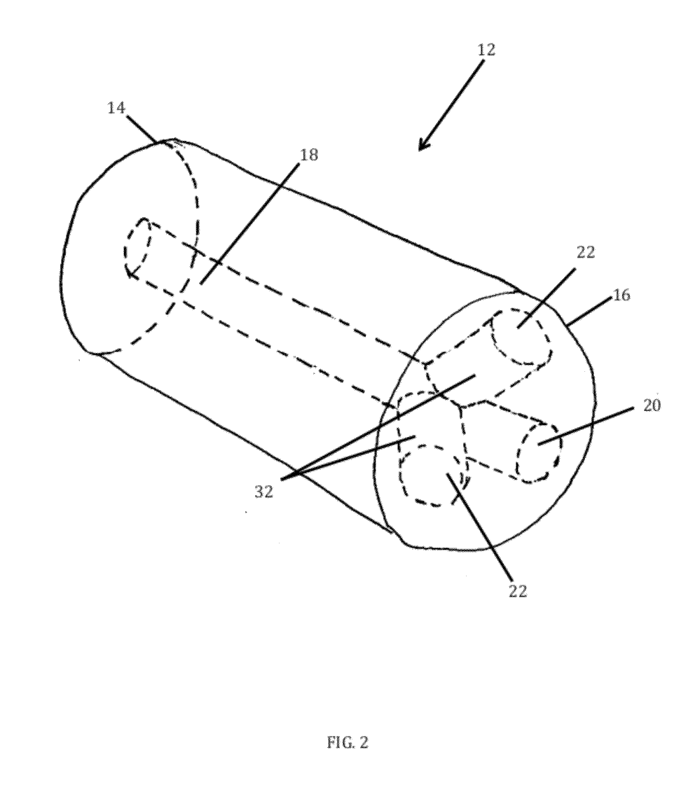

A first exemplary embodiment of a multi-port tip 12 is depicted in FIGS. 2-5. The elongate multi-port tip 12 is sized to be disposed in the ear canal of the user, and includes a first end 14 and a second end 16. In preferred embodiments, the tip 12 is molded from a relatively soft and flexible plastic. In other embodiments, hard materials could be used. A central channel 18 traverses the length of the tip 12 and conducts acoustic energy there through. The channel 18 originates at the first end 14 and terminates in the second end 16 at a central sound port 20.

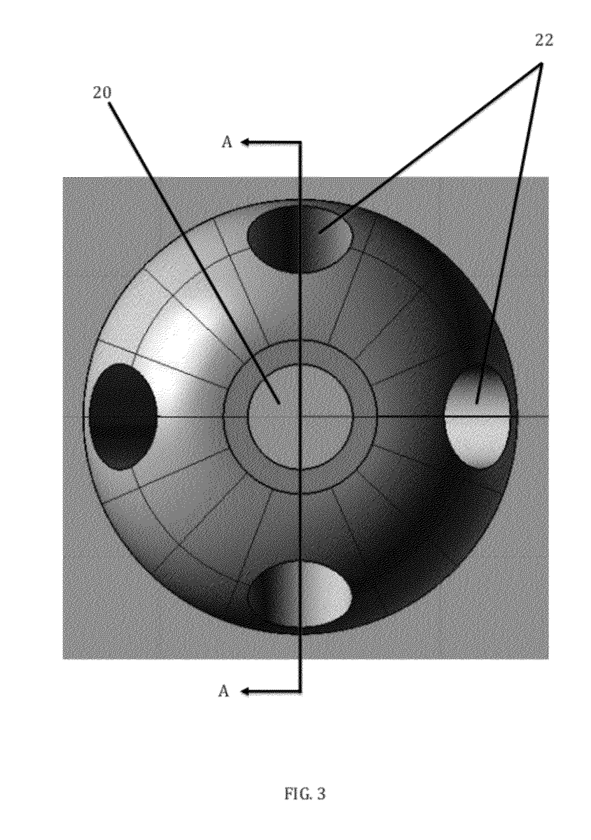

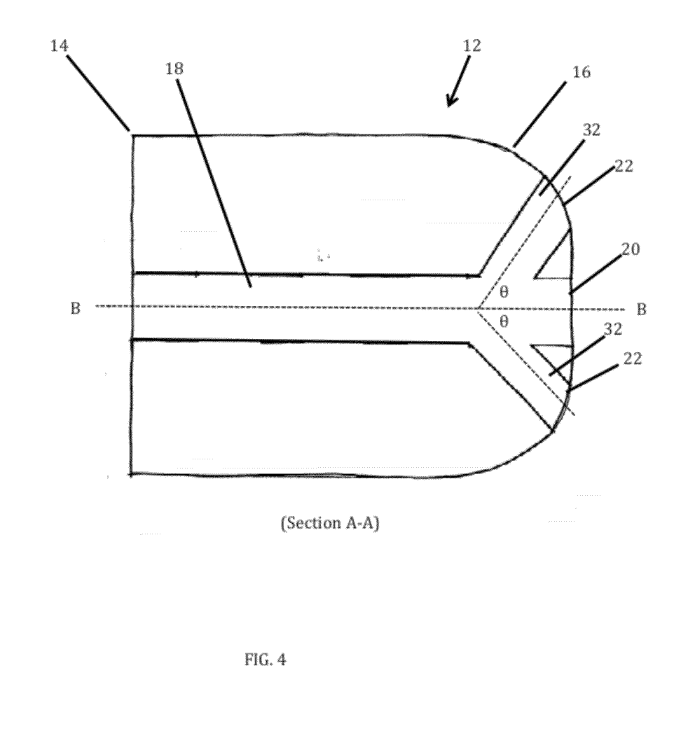

The tip 12 includes at least two sound ports—the central sound port 20 and at least one peripheral sound port 22—which are in direct communication with the central channel 18. The sound ports 20, 22 receive acoustic energy from the hearing aid device through the central channel 18 and conduct the acoustic energy into the ear canal of the user. As shown in FIGS. 2-5, the central sound port 20 is disposed substantially along a longitudinal axis (line “B-B” shown in FIG. 4) of the tip. Multiple peripheral sound ports 22 may be disposed around the central port 20, and may be placed in many configurations or angles with respect to the longitudinal axis of the tip 12. For example, the second sound ports 22 may be spaced radially around the central sound port 20. Additionally, the configuration may be altered to provide fewer or greater numbers of peripheral sound ports. For example, a tip 12 with one peripheral sound port 22 may have a different configuration from a tip having four peripheral sound ports (FIG. 3).

As shown in FIG. 4, the peripheral sound ports 22, like the central port 20, may stem from the central channel 18. This configuration enables sound to travel from the hearing aid device, into the central channel 18, and into the ear canal of the user via multiple sound ports 20, 22 simultaneously. If one of the ports (e.g., the central port) becomes blocked or partially blocked by ear wax or dead skin cells, other of the ports will likely not be blocked and loss in sound or sound quality will be minimal.

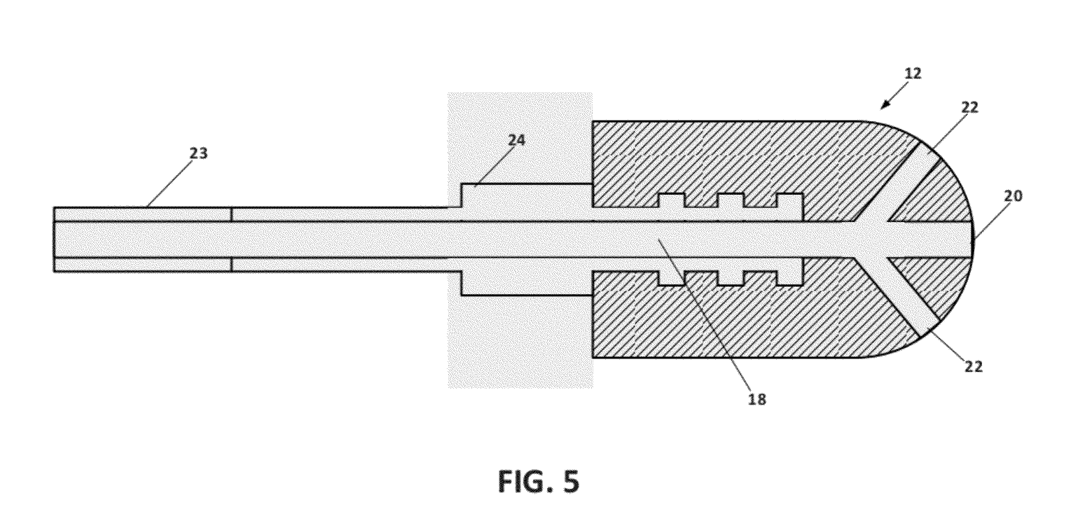

FIG. 5 illustrates a multi-port tip 12 and a connector member 24 for connecting a thin tube 23 of a hearing aid device to the tip 12. When the connector member 24 is connected to the tip 12, sound travels from the hearing aid device, through the thin tube 23, through the connector member 24, through the central channel 18, out of the sound ports 20 and 22 of the tip 12, and into the ear canal of the user.

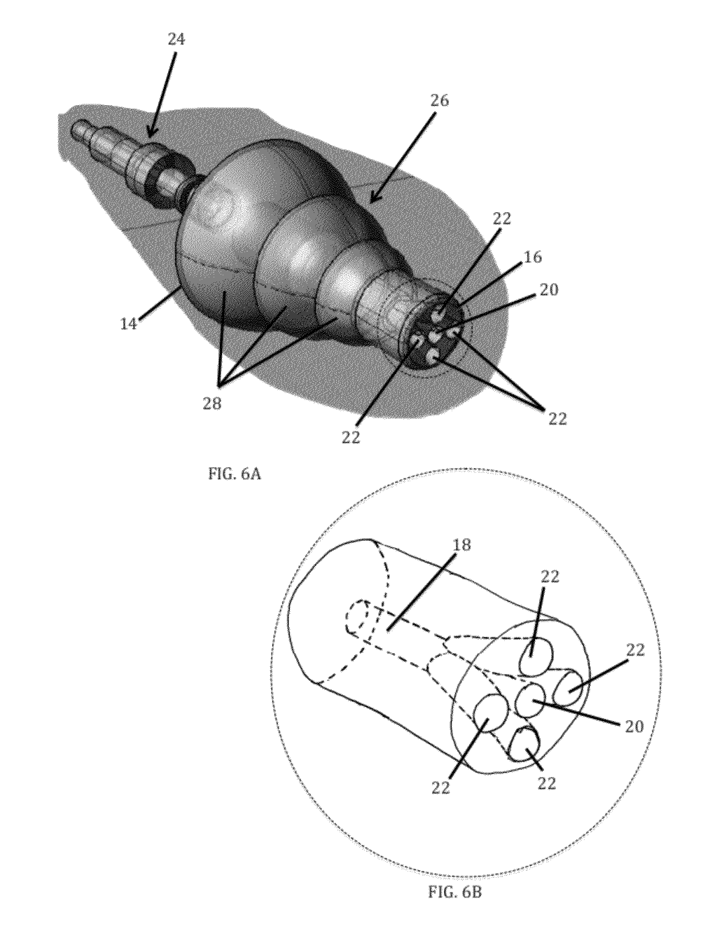

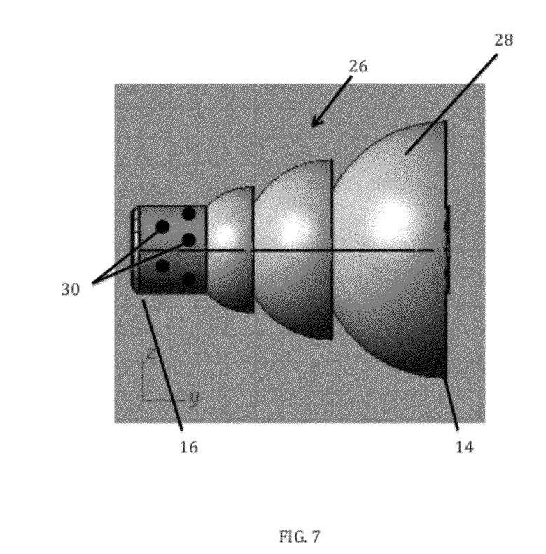

FIGS. 6 and 7 depict embodiments of a multi-port tip 26 having a flange assembly comprising a plurality of flange members 28. The flange members 28 enable the tip 26 to be used by a wide range of users, where each user may have varying ear canal sizes and shapes. As shown in FIG. 6, the tip 26 is encircled by multiple flange members 28 of various sizes, and where the smallest flange member is the innermost when inserted into the ear canal of the user. In addition to directing sound generated by the hearing aid device into the user's ear canal, the flange members 28 are designed to provide a noise excluding seal in the ear canal of the user. By providing multiple flanges 28 in varying sizes, the tip 26 is more likely to fit into and adequately seal the ear canals of various users who have ear canals of varying sizes and shapes.

FIG. 7 depicts an embodiment of a multi-port tip 26 having multiple peripheral sound ports 30 encircling the second end 16 of the tip. The peripheral sound ports 30 may be used in place of or in addition to other ports that have been previously described. Multiple peripheral sound ports 30 may be spaced radially around the second end 16. Preferably, all of the peripheral sound ports 30 are connected to and receive sound from the central channel 18 of the tip 26.

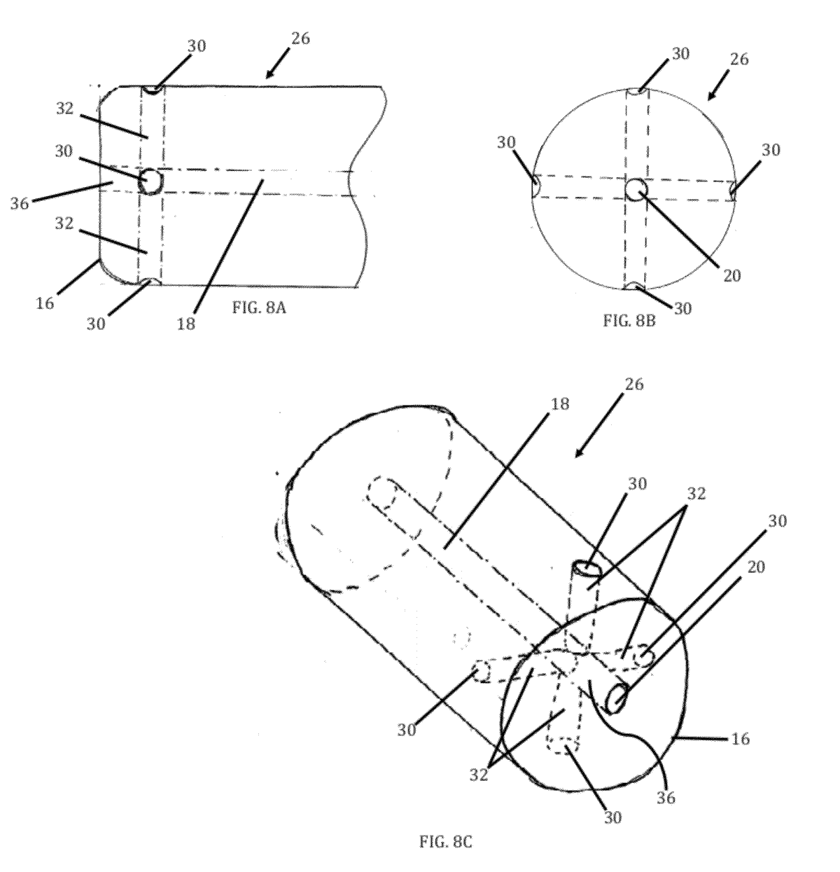

FIGS. 8A-8C depict an embodiment of a multi-port tip 26 wherein the second end 16 includes four peripheral sound port bores 32 connecting four peripheral sound ports 30 to the central channel 18. A central sound port bore 36 connects the central sound port 20 to the central channel 18. As shown, the peripheral sound port bores 32 are at an angle of about 90 degrees with respect to the longitudinal axis of the tip. Each of the four peripheral sound port bores 32 project outward from the central channel 18 and are radially offset from each adjacent peripheral sound port bore by an angle of about 90 degrees.

This configuration is particularly useful in maintaining the hearing aid device during its useful life and, in particular, in cleaning and servicing the tip. As noted earlier, ear wax deposits in the ear canal of a user tend to plug or to clog the ports in hearing aid tips. One method of removing this ear wax is to swab the tip with a thin brush or a wire or a monofilament line known as a plunger. The plunger is inserted into the clogged port opening and is used to remove any ear wax deposits that may have collected in the tip. One benefit of configuring the sound ports 30 as shown in FIGS. 8A-8C is that a plunger may be inserted into any one of the sound ports 30 and exit the tip through an opposing collinear sound port 30. This configuration allows any obstruction in the tip to easily be pushed out of the tip using the plunger via the opposite sound port.

Another technique for cleaning wax deposits from the tip involves flicking the end of the flexible tip with a finger. This dislodges the unwanted deposits from the sound ports.

In one embodiment, the tip 12 is an integral part of the thin tube 23 that carries sound from the BTE hearing aid into the ear canal. In this embodiment, the tip 12 and the thin tube 23 may be molded as a single piece, wherein the tip 12 is an extension of the thin tube 23. In one variation of this embodiment, the central sound port is a hole at the very end of the thin tube 23 and the peripheral sound ports are holes that are drilled or otherwise formed in the wall of the thin tube 23 near its end.

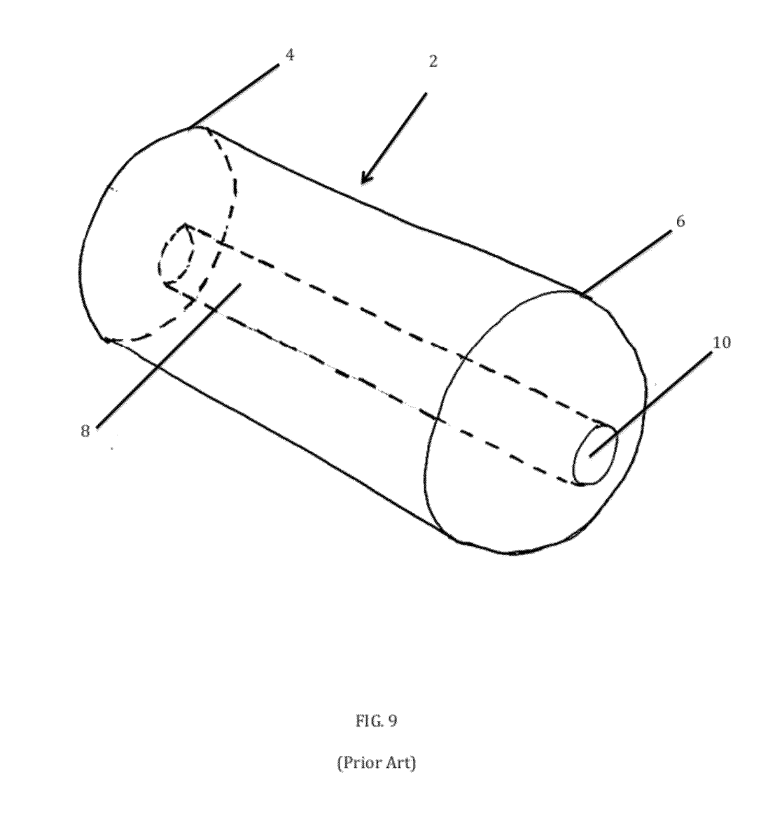

In all of the previously discussed embodiments, the positioning and angle of the peripheral sound ports 22 and 30 depend, at least in part on the configuration and geometry of the tip 26. In general, sound port bores may be placed at an angle θ (FIG. 4) ranging from about 90 degrees to about 10 degrees with respect to the longitudinal axis B-B. For example, in FIGS. 2-5, the peripheral port bores 32 are located in a hemispherical surface at the second end 16 of the tip at an angle θ of about 45 degrees. As another example, in FIG. 6, the tip 26 at the second end 16 is cylindrical, and the peripheral port bores 32 are disposed with the central sound port in the planar surface at the second end 16. In this embodiment, the bores 32 extend from the central channel 18 at a slight angle θ which is about 10-20 degrees. Finally, in FIGS. 9A and 9B, the peripheral sound ports 30 are shown having bores 32 that connect to the central channel 18 at angle θ of about 90 degrees. Thus, it will be appreciated that the invention is not limited to any particular angle of the sound port bores 32.

What is claimed is:

1. A tip operable for use in a hearing aid apparatus for imparting acoustic energy to a user for assistance in hearing, the tip comprising:
a channel portion operable for conducting acoustic energy and sized to be disposed in an ear canal of the user, the channel portion having a proximal end and a distal end and a central channel extending between the proximal end and the distal end; and
a tip portion disposed at the distal end of the channel portion, the tip portion sized to be disposed in the ear canal of the user, the tip portion including at least two sound ports in direct communication with the central channel of the channel portion to receive the acoustic energy therefrom, the at least two sound ports for conducting the acoustic energy from the central channel of the channel portion into the ear canal of the user.

2. The tip according to claim 1 wherein the at least two sound ports include a central sound port disposed substantially along a longitudinal axis of the tip and at least one peripheral sound port disposed adjacent the central sound port.

3. The tip of claim 2 wherein the at least one peripheral sound port comprises a plurality of peripheral sound ports disposed radially around the central sound port.

4. The tip of claim 3 wherein the plurality of peripheral sound ports comprise four peripheral sound ports that are substantially equally spaced radially around the central sound port.

5. The tip of claim 2 wherein the tip portion comprises a substantially hemispherical surface and the plurality of peripheral sound ports are disposed in the substantially hemispherical surface and are spaced radially apart around the central sound port.

6. The tip of claim 2 wherein the tip portion includes a substantially cylindrical surface and the plurality of peripheral sound ports are disposed in the substantially cylindrical surface and are spaced radially apart around the central sound port.

7. The tip of claim 2 wherein the at least one peripheral sound port includes a corresponding peripheral sound port bore connecting the at least one peripheral sound port to the central channel of the channel portion.

8. The tip of claim 7 wherein the peripheral sound port bore is disposed at an angle ranging from about 90 degrees to about 10 degrees with respect to the longitudinal axis of the tip.

9. A tip according to claim 1 wherein at least one of the at least two sound ports is a peripheral sound port disposed along a peripheral surface of the tip portion.

10. A tip according to claim 1 wherein the channel portion of the tip is an integral part of a thin tube that carries sound from the hearing aid apparatus into the ear canal of the user.

11. A tip according to claim 1 wherein the channel portion of the tip is operable to receive an end of a thin tube inserted therein, wherein the thin tube carries sound from the hearing aid apparatus to the tip.

12. A tip operable for use in a hearing aid apparatus for imparting acoustic energy to a user for assistance in hearing, the tip comprising:
a channel portion operable for conducting acoustic energy and sized to be disposed in an ear canal of the user, the channel portion having a proximal end and a distal end and a central channel extending between the proximal end and the distal end; and
a tip portion disposed at the distal end of the channel portion, the tip portion having a substantially hemispherical surface that is sized to be disposed in the ear canal of the user, the tip portion including a plurality of sound ports in direct communication with the central channel of the channel portion for receiving the acoustic energy therefrom and for conducting the acoustic energy from the central channel of the channel portion into the ear canal of the user, the plurality of sound ports including a central sound port disposed substantially along a longitudinal axis of the tip and a plurality of peripheral sound ports disposed in the substantially hemispherical surface of the tip portion at a substantially equal radial spacing around the central sound port,
wherein the tip portion includes a plurality of peripheral sound port bores connecting the plurality of peripheral sound ports to the central channel of the channel portion at an angle ranging from about 10 degrees to about 90 degrees with respect to the longitudinal axis of the tip, and a central sound port bore connecting the central sound port to the central channel of the channel portion.

13. A tip operable for use in a hearing aid apparatus for imparting acoustic energy to a user for assistance in hearing, the tip comprising:
a channel portion operable for conducting acoustic energy and sized to be disposed in an ear canal of the user, the channel portion having a proximal end and a distal end and a central channel extending between the proximal end and the distal end; and
a tip portion disposed at the distal end of the channel portion, the tip portion having a substantially cylindrical surface and sized to be disposed in the ear canal of the user, the tip portion including a plurality of sound ports in direct communication with the central channel of the channel portion for receiving the acoustic energy therefrom and conducting the acoustic energy from the central channel of the channel portion into the ear canal of the user, the plurality of sound ports including a central sound port disposed substantially along a longitudinal axis of the tip and four peripheral sound ports disposed in the substantially cylindrical surface of the tip portion at a substantially equal radial spacing around the central sound port,
wherein the tip portion includes four peripheral sound port bores connecting the four peripheral sound ports to the central channel of the channel portion at an angle of about 90 degrees with respect to the longitudinal axis of the tip, and a central sound port bore connecting the central sound port to the central channel of the channel portion.
